(19) United States
(12) Patent Application Publication
(10) Pub. No.: US 2024/0252834 A1
(43) Pub. Date: Aug. 1, 2024

(54) LIGHT WAVELENGTH APPLICATION SYSTEM

(71) Applicant: ICARO, Inc, Orlando, FL (US)

(72) Inventors: Michael Miller, Winter Garden, FL (US); Fredric Maxik, Melbourne, FL (US); Ran Zhou, Rockledge, FL (US)

(73) Assignee: ICARO, Inc., Orlando, FL (US)

(21) Appl. No.: 18/428,907

(22) Filed: Jan. 31, 2024

Related U.S. Application Data
(60) Provisional application No. 63/482,523, filed on Jan. 31, 2023.

(51) Int. Cl. A61N 5/06

(52) U.S. Cl. CPC A61N 5/06; A61N 2005/0626; A61N 2005/0645; A61N 2005/0652; A61N 2005/0659; A61N 2005/0667

(57) ABSTRACT

A treatment light emission system is provided. The system comprises a body member, one or more emission members, a control unit, and a power unit. The one or more emission members may be carried by the body member. The one or more emission members may selectively emit a treatment light. The control unit may be coupled in communication with the one or more emission members. The control unit may control the one or more emission members to emit the treatment light. The power unit may be coupled in communication with the control unit so that the control unit may be configured to provide and regulate power to the one or more emission members. The treatment light may comprise a wavelength of infrared (IR) light. The control unit may be separated and spaced apart from the body member.

### RELATED APPLICATIONS

This application claims priority under 35 U.S.C. § 119(e) of U.S. Provisional Patent Application Ser. No. 63/482,523 (Attorney Docket No. 6819.00002) filed on Jan. 31, 2023 and titled LIGHT WAVELENGTH APPLICATION SYSTEM, the entire contents of which is incorporated herein by reference except to the extent that the content therein conflicts with the content herein.

### FIELD OF THE INVENTION

The present invention relates to systems and methods for an emission device for emitting wavelengths of light for treating symptoms that arise from effects of pathogens, bacteria, parasites, fungi, viruses, and/or other cellular stress.

### BACKGROUND OF THE INVENTION

The use of wavelengths of light for therapeutic effects, alleviation, and for treatment of various symptoms that may be caused by a variety of underlying conditions and/or sources has shown promising use. Specifically, the use of particular wavelengths of light for treating physical ailments and the symptoms and sources thereof have shown to have beneficial effects upon application and exposure of an affected person or portion thereof having such symptoms and/or underlying negative condition. However, the prior art have failed to provide for a method or system implementing such treatment wavelengths of light in a way that is minimally distressing and also convenient from the perspective of the user and/or from the perspective of the subject/patient who is being exposed to such treatment by light wavelengths. Thus, there is a need left by the prior art for using, implementing, and providing for a device to expose users and/or patients to treatment wavelength(s) of light having a structure and/or by a process that is minimally user/patient distressing, minimally user/patient burdensome, and also convenient in use and in its application for users and patients.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to a treatment light emission system comprising a body member, one or more emission members, a control unit, and a power unit. The one or more emission members may be carried by the body member. The one or more emission members may selectively emit a treatment light. The control unit may be coupled in communication with the one or more emission members. The control unit may also control the one or more emission members to emit the treatment light. The power unit may be coupled in communication with the control unit so that the control unit may be configured to provide and regulate power to the one or more emission members. The treatment light may comprise a wavelength of infrared (IR) light. The control unit may be separated and spaced apart from the body member. The control unit may be selectively wired communication with the one or more emission members and with the power unit.

The one or more emission members may comprise a plurality of emission members carried by the body member that may each selectively emit the treatment light. The control unit may selectively control the plurality of emission members to cause one or more of the emission members to emit the treatment light. The one or more emission members may include a lighting filament and/or a light emitting diode (LED).

Some embodiments of the present invention may include a conversion coating that may be positioned the one or more emission members. The treatment light emitted by the one or more emission members may have a first wavelength. The conversion coating may filter the first wavelength of light emitted from the one or more emission members to a converted treatment light that may have a second wavelength of light that may be different from the first wavelength of light.

Some embodiments of the present invention may include a monitor unit. The monitor unit may be positioned in proximity to the body member. The monitor unit may be in communication with the control unit. The monitor unit may be configured to detect at least a portion the treatment light which may be defined as a detected light. The monitor unit may determine characteristics of the detected light. The monitor unit may emit a light detection signal to the control unit. The control unit may cause the one or more emission members to take a predetermined action responsive to the light detection signal.

The characteristics of the detected light may be defined as one or more of power, intensity, relative location, amplitude, and wavelength of the detected light. The light detection signal may contain computer readable information that may include one or more of power, intensity, relative location, amplitude, and wavelength of the detected light. The predetermined action responsive to the light detection signal may comprise one or more of ceasing emission of the treatment light, decrease the intensity of the treatment light, increase the intensity of the treatment light, and/or change a location of the treatment light.

Some embodiments of the present invention may include an application member that may be carried by the body member. The application member may emit the treatment light after being charged by one or more wavelengths of light. The one or more wavelengths of light used to charge the application member may include one or more of an ultraviolet light having a wavelength of between about 253.7 to 275 nanometers and a blue light. The application member may emit the treatment light for a period of time after the application member is charged by the one or more wavelengths of light. The treatment light may have a wavelength between about 1038 and 1078 nanometers wavelength.

Some embodiments of the present invention may be direct to a treatment light emission system comprising a body member and an application member that may be carried by the body member. The application member may emit a treatment light. The treatment light may comprise a wavelength of infrared (IR) light. The application member may emit the treatment light after being exposed to and/or charged by one or more wavelengths of light that may be defined as charging light. The application member may emit the treatment light for an extended period of time after the application member is exposed to the charging light.

Some embodiments of the present invention may include a charging unit. The charging unit may provide the charging light. The charging unit may be separated and spaced apart from the body member. The charging light may comprise ultraviolet light and/or blue field light. The treatment light may have a wavelength of between about 1038 and 1078 nanometers. The application member may comprise a long-latency phosphor.

Some embodiments of the present invention may be directed to a treatment light emission system comprising a body member, an application member, and a charging unit. The application member may be carried by the body member. The application may be configured to emit a treatment light. The charging unit may provide at one or more wavelengths of light which may be defined as charging light. The application member may emit the treatment light after being exposed to the charging light. The treatment light may comprise a wavelength of infrared (IR) light. The charging unit may be separated and spaced apart from the body member. The application member may emit the treatment light for a period of time after the application member is exposed to the charging light.

The charging light may comprise ultraviolet (UV) light and/or blue field light. The treatment light may have a wavelength between about 1038 and 1078 nanometers. The application member may comprise a long-latency phosphor.

### BRIEF DESCRIPTION OF THE DRAWINGS

FIG. 1 is a perspective view of a light emission system according to an embodiment of the present invention having a body member in the shape of a blanket and an emission member comprising fiber optic cable.

FIG. 2 is another perspective view of a light emission system according to an embodiment of the present invention having a body member in the shape of clothing.

FIG. 3 is a perspective view of the light emission system illustrated in FIG. 1, without a body member.

FIG. 4 is a perspective view of a light emission system according to an embodiment of the present invention having emission member comprising light emitting diodes.

FIG. 5 is a perspective view of a body member including an application member, of a light emission system according to an embodiment of the present invention.

FIG. 6 is a schematic view of the body member illustrated in FIG. 5 being charged by a charging unit.

FIG. 7 is a perspective view of the body member illustrated in FIG. 5 showing a release of emitted waves.

FIG. 8 is a schematic diagram of the light emission system according to FIG. 1.

FIG. 9 is a schematic diagram of the light emission system according to FIG. 6 and including a charging unit.

### DETAILED DESCRIPTION OF THE INVENTION

The present invention will now be described more fully hereinafter with reference to the accompanying drawings, in which preferred embodiments of the invention are shown. This invention may however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

An embodiment of the invention, as shown and described by the various figures and accompanying text, provides a light emission system 100 that may be used to treat, aid, affect, and/or assist individuals in dealing with the symptoms of a number of ailments including, but not limited to, cuts, bruises, wounds, diseases, afflictions, circulatory system problems, breathing problems, viral infections, bacterial infections, fungal infections, sicknesses, discomforts, and/or cellular stresses. The light emission system 100 may advantageously provide for individuals to have a comfortable and/or simple way of applying certain wavelengths of light to the individuals' person and/or to portions of the individuals' person. As will be described in more detail below, the light emission system 100 may emit certain wavelengths of light in a comfortable manner to treat an individual's person. Those skilled in the art will appreciate that the light that may be emitted by the light emission system may be any type of light including, but not limited to infrared light, ultraviolet light, blue light, or any other type of light as understood within the art.

For instance, the light emission system 100 may comprise a blanket used to cover an individual's person and that is adapted to emit light to treat the individual's ailments, wounds, symptoms, and/or other cellular stressors. The light emission system 100 may also comprise athletic tape, socks, or a bandage that may be coated with a certain coating that is charged with ultraviolet light or blue light, which will then discharge and emit infrared light for an extended period of time and can be worn by an individual to have doses of infrared light applied to those portions of the individual's person.

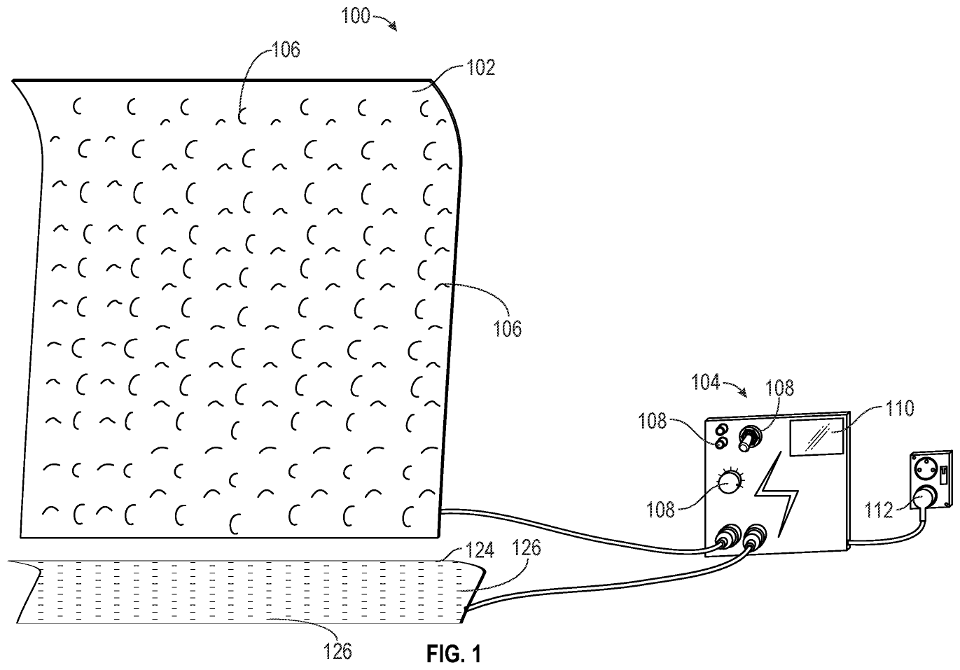

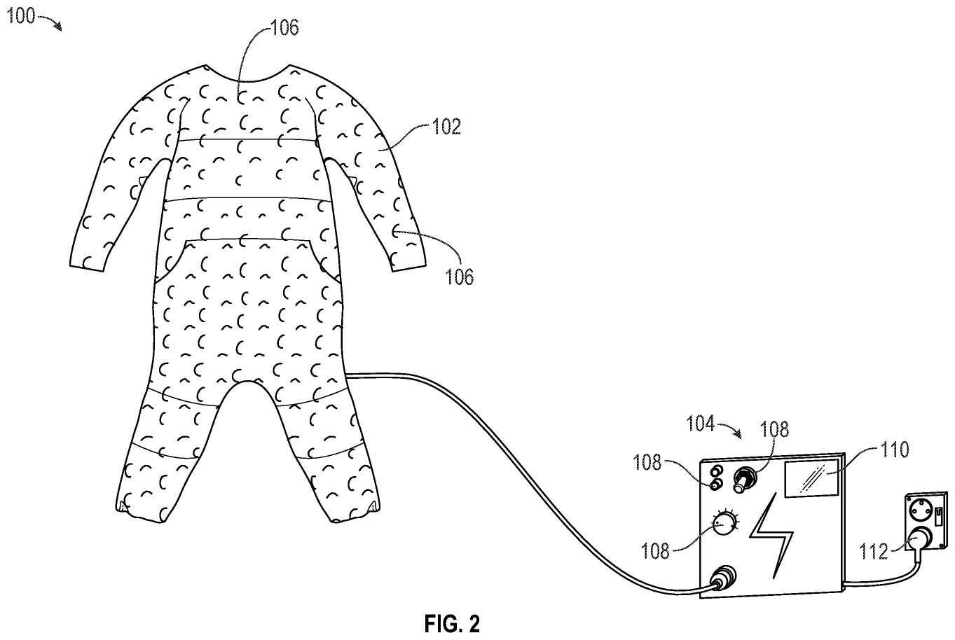

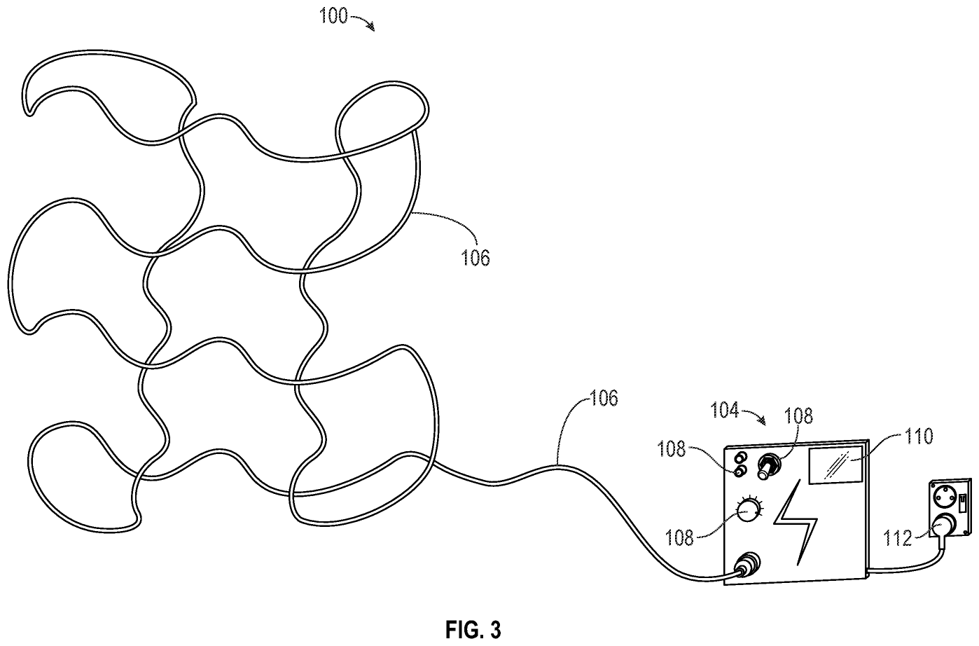

The embodiments of the present invention will now be described in greater detail. Initially referring to FIGS. 1-3 and 8, the light emission system 100 may include a body member 102 and a control unit 104. The body member 102 may comprise any kind of fabric, material, or clothing, such as, without limitation, blankets, covers, pads, bandages, tapes, silicones, sheets, jump-suits, shirts, pants, socks, hats, blankies, onesies, pajamas, and/or undergarments. The body member 102 may comprise a conversion coating, such as a light conversion coating, that may filter and/or transform light that is emitted from a source light. The light that is emitted from the source light and passed through the conversion coating may be referred to as converted light. The conversion coating may filter out certain ranges of wavelengths of light, convert one or more wavelength(s) of light to one or more different wavelength(s) of light, reduce the intensity of light that passes through the conversion coating, and/or absorb all or some particular wavelengths of light. For example, without limitation, the conversion coating may filter out all or certain portions of blue light, infrared light, ultraviolet light, and/or visible light of the light that passes through the conversion coating. In another example, and also without limitation, the conversion coating may absorb and/or transform one or more wavelengths of ultraviolet light, and the conversion coating may emit one or more wavelengths of infrared light after absorbing and/or transforming the ultraviolet light.

The system 100 may include one or more emission members 106. As noted above, the emission member 106 may also, in some embodiments, be referred to as source lights. The emission member(s) 106 may be carried by and/or attached to the body member 102. The emission member(s) 106 may comprise an elongated light emission line, such as fiber optic cable and/or side emitting fiber optic cable, which may be intertwined with, or woven throughout, the body member 102. Those skilled in the art will notice and appreciate that any standard or non-standard filament capable of facilitating light and/or infrared light may be used as the emission member(s) 106 while still accomplishing all the goals, features, and advantages of an embodiment of the present invention. The emission member(s) 106 may also comprise additional structures to enhance the emission of light, such as, prismatic structures, luminescent micro ceramics, and metal/metallic coatings.

The emission members 106 may be configured to generate, accommodate, facilitate, and/or radiate light including infrared light. Preferably, the infrared light is in the range of about 1058-1078 nanometers of wavelength, but those skilled in the art will appreciate that the infrared light may have any wavelength(s) within the infrared wavelength range. The emission member(s) 106 may be coupled in communication with the control unit 104. The control unit 104 may be configured to provide and regulate power of the emission members 106.

In some embodiments of the present invention, the emission member(s) 106 may comprise fiber optic cable or another lighting filament that do not generate light, the control unit 104 may be configured to provide and/or generate the source of light that may be directed through and facilitated by the emission member(s) 106, i.e., source light, which may be carried by and radiated out from the emission member(s) 106. The source of light provided by the control unit 104 may be infrared light in the range of about 1058-1078 nanometers of wavelength. However, those skilled in the art will notice and appreciate that the emission members 106 may be configured to facilitate and/or radiate any wavelength(s) of light, and the control unit 104 may be configured to provide and/or generate any wavelength of light. The other wavelengths of light may include, without limitation, ultraviolet light, visible light, and any other wavelength(s) of infrared light.

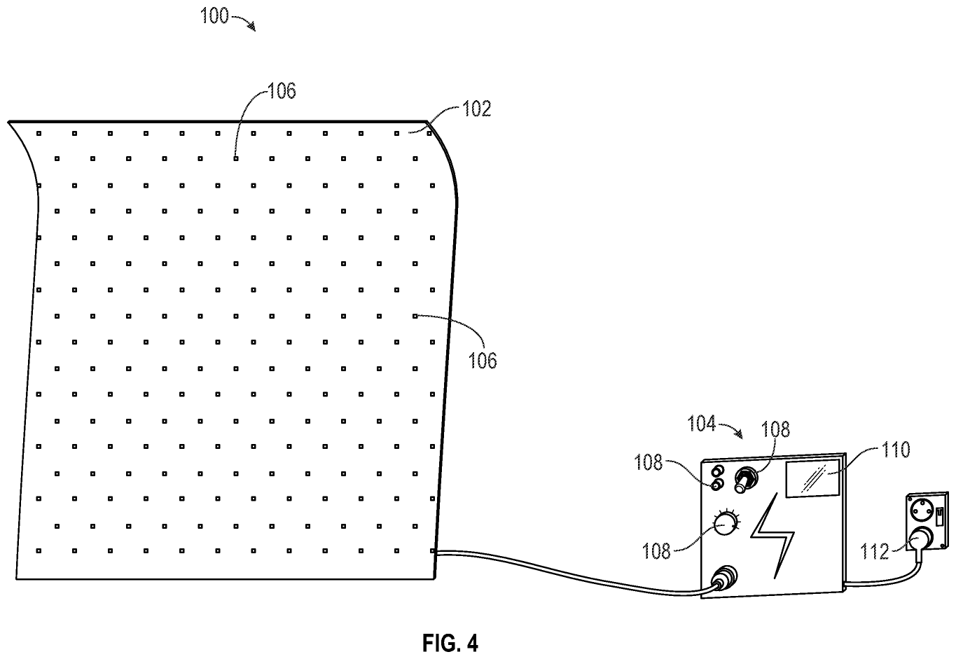

Now additionally referring to FIG. 4, the emission members 106 may also comprise light generators such that the emission members 106 generate the light rather than facilitate/carry/radiate the light. The light generators may comprise light emitting diodes that may be configured to generate one or more wavelengths of light, including infrared light in the range of about 1058-1078 nanometers of wavelength. More specifically, the light emitting diodes may be configured to first generate infrared light in the range of about 1038-1058 nanometers of wavelength so that when the light emitting diodes warm up and reach a predetermined operating temperature, the light emitting diodes may then generate infrared light in the range of about 1058-1078 nanometers of wavelength. However, the light emitting diodes and light generators may be configured to generate any wavelength(s) of light including, without limitation, ultraviolet light, visible light, and any other wavelength(s) of infrared light.

The emission member(s) 106 may be configured so that only a portion of the emission member(s) 106 will generate and/or emit light. For example, without limitation, more than one emission member 106 may be coupled in communication with the control unit 104 and/or the power unit 156 so that a select number of the emission members 106 may be selectively caused to generate and/or emit light. This may be advantageous on an occasion where the body member 102 of the system 100 comprises a blanket or a jump-suit (or any similar article suitable to be worn by a user) is on an individual so that a portion of the emission member(s) 106 can be activated to generate and/or emit light on a select area of the individual's person, such as, the legs, arms, feet, hands, and chest.

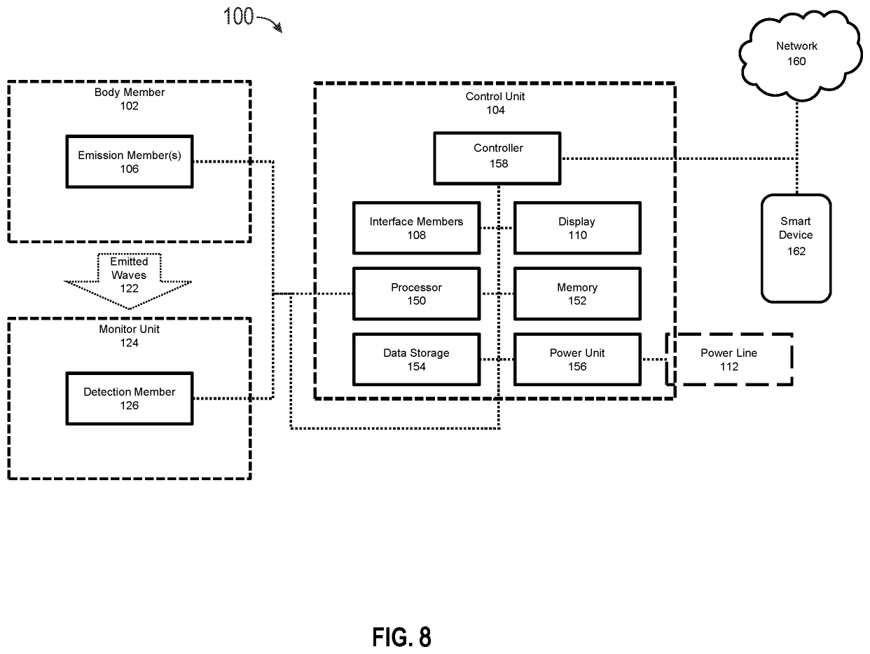

Now referring specifically to FIGS. 1 and 8, an embodiment of the present invention may include a monitor unit 124 having one or more detection members 126. The monitor unit 124 and/or detection members 126 may be coupled in communication with the control unit 104. The monitor unit 124 and/or detection member(s) 126 may be configured to detect one or more wavelengths of light, including infrared light in the range of about 1058 to 1078 nanometers in wavelength. The power, intensity, relative location, amplitude, and/or wavelength of the wavelengths of light detected by the monitor unit 124 and/or the detection member(s) 126 may be determined by the monitor unit 124, the detection member(s) 126, and/or the control unit 104.

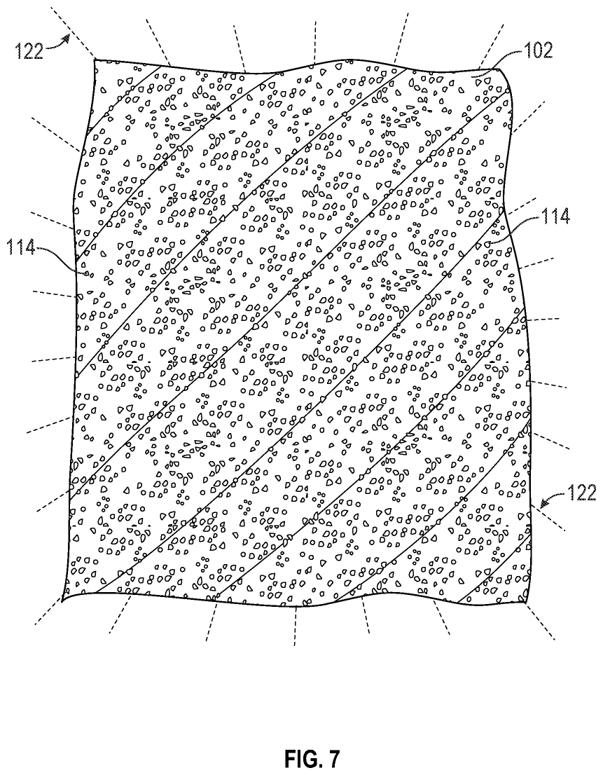

The monitor unit 124 and/or the detection member(s) 126 may be configured to emit a light detection signal relating to light detected in emission waves 122 (FIG. 7) from the emission members 106 and/or the body member 102. The light detection signal may contain computer readable information including the power, intensity, amplitude, wavelength, and/or location relative to the monitor unit 124 and/or the detection member(s) 126. The control unit 104 may be configured to receive the light detection signal and take a predetermined actioned upon receipt. For the example, upon the control unit 104 receiving the light detection signal, the control unit 104 may cause the emission member(s) 106 to stop generating/emitting light, decrease the intensity of the light being generated/emitted, and/or increase the intensity of the light being generated/emitted, which may be done to a portion or a select number of the light emission member(s) 106.

The monitor unit 124 may also be configured to detect the presence and/or proximity of an individual and/or a life form (such as a pet, for example) that may be positioned upon or in close proximity to the monitor unit 124. The monitor unit 124 may be configured to emit a presence detection signal that may be related to the presence of the individual or life form that is positioned upon or in close proximity to the monitor unit 124. The control unit 104 may be configured to receive the presence detection signal and take a predetermined actioned upon receipt. For the example, upon the control unit 104 receiving the presence detection signal, the control unit 104 may cause the emission member(s) 106 to stop generating/emitting light, decrease the intensity of the light being generated/emitted, and/or increase the intensity of the light being generated/emitted. Although the monitor unit 124 is described as having the capability to detect presence, those skilled in the art will appreciate that the system 100 may also include a separate presence detection device.

Continuing to refer to FIG. 8, the control unit 104 may include a controller 158, a processor 150, one or more interface members 108, a display 110, a processor 150, memory 152, data a storage 154, a power unit 156, and a power line 112. The power unit 156 may be in communication with the power line 112. The power unit 156 may receive and/or regulate power received from the power line 112, which the power line 112 may receive the power from a power source, such as, a power outlet. The power unit 156 may also, or alternatively, comprise a power storage unit or battery that may be replaceable and/or rechargeable via the power line 112.

The power unit 156 may be configured to send and/or regulate electrical power to the emission members 106, the detection members 126, the data storage 154, the processor 150, the interface members 108, the display 110, the memory 152, and/or the controller 158. In some embodiments of the present invention, the power unit 156 may be configured to generate one or more wavelengths of light that may be sent to and/or through the emission members 106. The light generated by the power unit 156 may include infrared light in the range of about 1058 to 1078 nanometers in wavelength.

The interface members 108 may be positioned on an outer portion of the control unit 104. The interface members 108 may include one or more buttons, knobs, switches, sliders, and/or keypads that may allow for user input. The input members 108 may emit input signals related to inputs made by a user. The input signals may be received by the controller 158 and/or the processor 150 and the controller 158 and/or the processor 150 may cause a predetermined action to take place related to the input signal received. For the example, without limitation, upon the controller 158 and/or the processor 150 receiving an input signal, the controller 158 and/or the processor 150 may cause the emission member(s) 106 and/or the power unit 156 to stop generating/emitting light, decrease the intensity of the light being generated/emitted, and/or increase the intensity of the light being generated/emitted, which may be done to a portion or a select number of the light emission member(s) 106 based upon the input signal.

The display 110 may be positioned on an outer portion of the control unit 104. The display 110 may be in communication with the controller 158 and/or the processor 150. The display 110 may be configured to display various statuses and information regarding the operation of the system 100. Specifically, the display 110 may display the status and operation of emission members 106, the power unit 156, the detection members 126, the monitor unit 124, the interface members 108, and/or error messages.

The data storage 154 may be carried by the control unit 104. The data storage 154 may be in communication with the memory 152, the processor 150, the controller 158, and the power unit 156. The data storage 154 may be configured to send, receive, read, write, delete, and store computer readable code, information, instructions, and data. The data storage 154 may comprise a hard disk drive, a solid state drive, flash drive, and/or nonvolatile computer data storage or any other device capable of acting as the data storage 154 as understood by those skilled in the art.

The memory 152 may be carried by the control unit 104. The memory 152 may be in communication with the data storage 154, the processor 150, the controller 158, and the power unit 156. The data storage 154 may be configured to send, receive, read, write, delete, and store computer readable code, information, instructions, and data. The memory 152 may comprise a hard disk drive, a solid state drive, flash drive, random access memory (RAM), nonvolatile computer readable memory, nonvolatile computer readable memory, and/or any other device capable of acting as the memory 152 as understood by those skilled in the art.

The processor 150 may be carried by the control unit 104. The processor 150 may be in communication with the controller 158, the network 160, the smart device 162, the interface members 108, the display 110, the memory 152, the power unit 156, the data storage 154, the emission members 106, the monitor unit 124, and/or the detection members 126. The processor 150 may be configured to receive, send, read, write, delete, store, compute, execute, process, compile, and/or perform computer readable instructions, code, commands, and/or information. The processor 150 may be configured to monitor and/or control the power unit 156, the memory 152, the display 110, the interface members 108, the data storage 154, the controller 158, the emission members 106, the monitor unit 124, and/or the detection members 126. The processor 150 may comprise a central processing unit (CPU), a graphics processing unit (GPU), a microcontroller, a microprocessor, an embedded processor, an application specific integrated circuit (ASIC), and/or any other computation device that may be used as the processor 150 as understood by those skilled in the art.

The controller 158 may be carried by the control unit 104. The controller 158 may be in communication with a smart device 162. The communication connection between the controller 158 and the smart device 162 may be via a network 160. The controller 158 may also be in communication with the interface members 108, the display 110, the processor 150, the memory 152, the data storage 154, the power unit 156, the emission members 106, the monitor unit 124, and/or the detection members 126. The controller 158 may be configured to receive instruction signals from the smart device 162. The smart device 162 may be operable by a user to send the instruction signals to the controller 158. The controller 158 may take a predetermined action based upon the instruction signal received from the smart device 162.

For example, without limitation, upon the controller 158 receiving an instruction signal from a smart device 162, the controller 158 may: cause the emission member(s) 106, control unit 104, and/or the power unit 156 to start/stop generating/emitting light; cause a decrease the intensity of the light being generated/emitted by the emission member(s) 106, control unit 104, and/or the power unit 156; and/or cause an increase the intensity of the light being generated/emitted by the emission member(s) 106, control unit 104, and/or the power unit 156. Any of the aforementioned predetermined actions taken by the controller 158 upon receipt of an instruction signal may be done only to a portion or a select number of the light emission member(s) 106.

The controller 158 may also include being configured to monitor the power unit 156, the emission members 106, and the detection members 126. The controller 158 may be configured to send a status signal to a smart device 162, which may be sent via the network 160. The status signal may relate to the current status, operation, power input, power output, and/or detected variables by the power unit 156, the emission members 106, and/or the detection members 126. The smart device 162 may receive the status signal for the status signal to be displayed thereon the smart device 162.

The communication between the controller 158 and the smart device 162 may be via wi-fi, Bluetooth, near field communication (NFC), ad hoc wi-fi, cellular data, cellular network, satellite network, universal serial bus (USB), serial ATA (SATA), and/or any other kind of wired or wireless communication connection as understood by those skilled in the art.

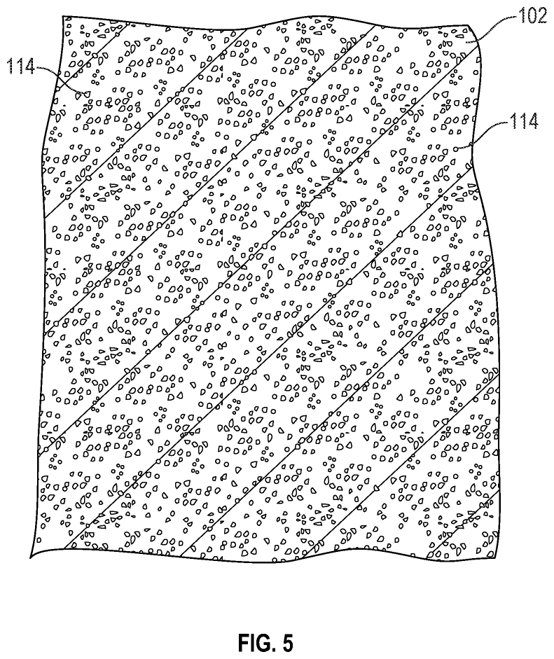

Now referring to FIGS. 5-7 and 9, some embodiments of the present invention may include a body member 102 and an application member 114. As mentioned further above, the body member 102 may comprise a number of objects and materials including, without limitation, clothing, shirts, pants, hats, blankets, bandages, wraps, tapes, athletic tapes, cotton, nylon, wool, polyester, silicone, and/or any other object or material as understood by those skilled in the art.

The application member 114 may be attached to the body member 102. The application member 114 may be uniformly spread upon, coated upon, and/or embedded within the body member 102, e.g., woven through portions of the body member. The application member 114 may also be laminated onto a body member 102. The application member 114 may comprise a chemical and/or compound, such as phosphor, which may more specifically be a long latency phosphor. The application member 114 may be configured to be exposed to and/or charged with a wavelength of light. The wavelength(s) of light that charge the application member 114 may be ultraviolet light (such as, for example, ultraviolet light having a wavelength of about 253.7 to 275 nanometers) and/or blue light.

The application member 114 may be configured to emit light, which may be infrared light, after the application member 114 has been charged. Once the application member 114 is charged, the application member 114 may emit the light/infrared light for an extended period of time, such at 24 to 48 hours. To charge the application member 114, and embodiment of the present invention may include a charging unit 116. The charging unit 116 may comprise a bag, sack, bin, box, or other container. The charging unit 116 may also comprise a housing, a power unit 156, a charging unit controller 128, a charging unit interface 118, a display 110, and one or more generators 130. The power unit 156, the charging unit controller 128, and charging unit interface 118, the display 110, and the generators 130 may be carried by the charging unit 116, which may be by the housing of the charging unit 116. The charging unit 116 and/or the housing of the charging unit 116 may include an access so that objects may be placed within and taken out from the charging unit 116. The access may comprise a door, hatch, opening, or passageway.

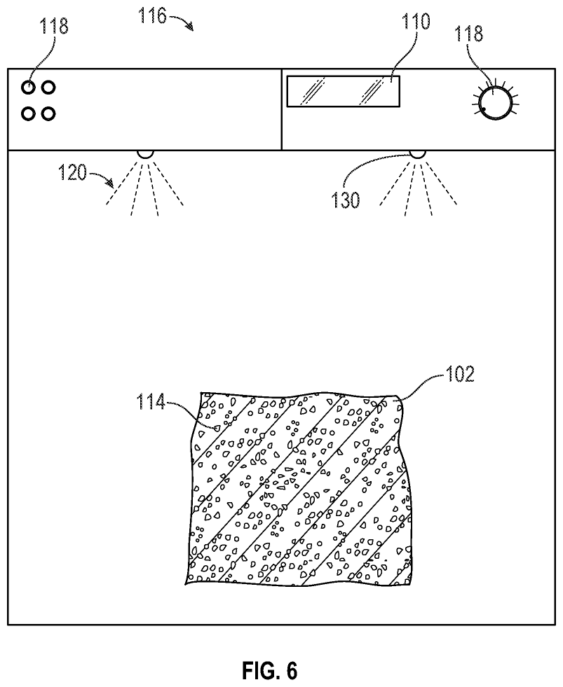

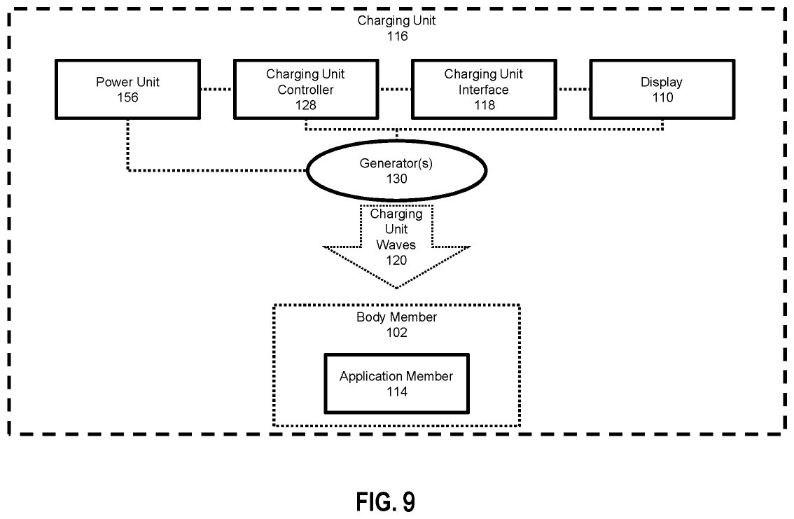

The generators 130 may be coupled in communication with the power unit 156, the charging unit controller 128, and/or the charging unit interface 118. The generators 130 may be configured to emit one or more wavelengths of light, as illustratively shown in FIGS. 6 and 9 as the charging unit waves 120. The charging unit waves 120 may comprise ultraviolet light, or blue field light. Ultraviolet light charging unit waves 120 may comprise ultraviolet light having a wavelength of about 253.7 to 275 nanometers. The generators 130 may comprise one or more of incandescent bulbs, light emitting diodes, fluorescent bulbs, retile basking bulbs, UVA lights, and/or UVB lights.

The application member 114 may absorb, convert, and/or transform the charging unit waves 120 and emit emitted waves 122, as illustratively shown in FIG. 7. Specifically, the application member 114 may absorb, convert, and/or transform the ultraviolet waves and/or blue light from the charging unit waves 120 and then emit emitted waves 122 that may comprise infrared light waves. Those skilled in the art will notice and appreciate that the application member 114 allows for wirelessly emitting infrared light after the application member 114 has been charged by the charging unit 116, which allows for increased portability and convenience compared to needing a constant source of power to emit infrared light.

The charging unit controller 128 may be coupled in communication with the power unit 156, the charging unit interface 118, the display 110, and the generators 130. The charging unit controller 128 may be configured to monitor, control, manage, send data, receive data, and/or maintain the power unit 156, the charging unit interface 118, the display 110, and the generators 130. The charging unit controller 128 may comprise a processor, microcontroller, embedded processor, memory, data storage, and/or any other computer-implementing device that may be used as the charging unit controller 128 as understood by those skilled in the art.

The charging unit interface 118 may be positioned on the charging unit 116 or the housing of the charging unit 116. The charging unit interface may be coupled in communication with the power unit 156, the charging unit controller 128, the display 110, and the generators 130. The charging unit interface 118 may be configured to be operable by a user to control and/or operate the charging unit 116. For example, without limitation, the charging unit interface 118 may be operable by a user to control power supplied by the power unit 156, the light emitted by the generators 130, and the information displayed by the display 110. The charging unit interface 118 may comprise one or more of buttons, switches, knobs, turn dials, a keypad, and/or levers. The charging unit interface 118 may be configured to emit an interface control signal upon a user operating the charging unit interface 118.

The display 110 may be coupled in communication with the charging unit interface 118, the charging unit controller 128, and the power unit 156. The display 110 may be configured to display information relating to data received from the charging unit interface 118 and/or the charging unit controller 128.

The power unit 156 may be coupled in communication with the charging unit controller 128, the charging unit interface 118, the display 110, and the generators 130. The power unit 156 may be configured to regulate and/or supply power to the charging unit controller 128, the charging unit interface 118, the display 110, and the generators 130. The power unit 156 may include one or more of batteries, replaceable batteries, rechargeable batteries, power supplies, voltage regulators, circuit breakers, fuses, and/or any other power device as understood by those skilled in the art.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the description of the invention. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

What is claimed is:

1. A treatment light emission system comprising:
a body member;
at least one emission member carried by the body member to selectively emit a treatment light;
a control unit coupled in communication with the at least one emission member to control the at least one emission member to emit the treatment light; and
a power unit coupled in communication with the control unit so that the control unit is configured to provide and regulate power to the at least one emission member;
wherein the treatment light comprises at least one wavelength of infrared (IR) light;
wherein the control unit is separated and spaced apart from the body member; and
wherein the control unit is selectively wired communication with the at least one emission member and with the power unit.

2. The system of claim 1, wherein the at least one emission member comprises a plurality of emission members carried by the body member to selectively emit the treatment light; and wherein the control unit selectively controls the plurality of emission members to cause at least one of the plurality of emission members to emit the treatment light.

3. The system of claim 1, wherein the at least one emission member comprises of at least one of a lighting filament and a light emitting diode (LED).

4. The system of claim 1, further comprising a conversion coating positioned on the at least one emission member; wherein the treatment light emitted by the at least one emission member has a first wavelength; and wherein the conversion coating filters the first wavelength of light emitted from the at least one emission member to a converted treatment light having a second wavelength of light that is different from the first wavelength of light.

5. The system of claim 1, further comprising a monitor unit positioned in proximity to the body member in communication with the control unit; wherein the monitor unit is configured to detect at least a portion the treatment light to be defined as a detected light and determine characteristics of the detected light; wherein the monitor unit emits a light detection signal to the control unit; and wherein the control unit causes the at least one emission member to take a predetermined action responsive to the light detection signal.

6. The system of claim 5, wherein the characteristics of the detected light are defined as at least one of power, intensity, relative location, amplitude, and wavelength of the detected light; and wherein the light detection signal contains computer readable information that includes at least one of power, intensity, relative location, amplitude, and wavelength of the detected light.

7. The system of claim 6 wherein the predetermined action responsive to the light detection signal is at least one of ceasing emission of the treatment light, decrease the intensity of the treatment light, increase the intensity of the treatment light, and change a location of the treatment light.

8. The system of claim 1, further comprising an application member carried by the body member to emit the treatment light after being charged by at least one wavelength of light.

9. The system of claim 8, wherein the at least one wavelength of light used to charge the application member is at least one of an ultraviolet light having a wavelength of between about 253.7 to 275 nanometers and a blue field light.

10. The system of claim 8, wherein the application member emits the treatment light for a period of time after the application member is charged by the at least one wavelength of light.

11. The system of claim 1, wherein the treatment light has a wavelength between about 1038 and 1078 nanometers wavelength.

12. A treatment light emission system comprising:
a body member; and
an application member carried by the body member to emit a treatment light;
wherein the treatment light comprises at least one wavelength of infrared (IR) light;
wherein the application member emits the treatment light after being at least one of exposed to and charged by at least one wavelength of light defined as charging light; and
wherein the application member emits the treatment light for an extended period of time after the application member is exposed to the charging light.

13. The system of claim 12, further comprising a charging unit to provide the charging light; wherein the charging unit is separated and spaced apart from the body member.

14. The system of claim 12, wherein the charging light comprises at least one of ultraviolet light and blue field light.

15. The system of claim 12, wherein the treatment light has a wavelength of between about 1038 and 1078 nanometers.

16. The system of claim 12, wherein the application member comprises a long-latency phosphor.

17. A treatment light emission system comprising:
a body member;
an application member carried by the body member to emit a treatment light; and
a charging unit to provide at least one wavelength of light defined as charging light;
wherein the application member emits the treatment light after being exposed to the charging light;
wherein the treatment light comprises at least one wavelength of infrared (IR) light;
wherein the charging unit is separated and spaced apart from the body member; and
wherein the application member emits the treatment light for a period of time after the application member is exposed to the charging light.

18. The system of claim 17, the charging light comprises at least one of ultraviolet (UV) light and blue field light.

19. The system of claim 17, wherein the treatment light has a wavelength between about 1038 and 1078 nanometers.

20. The system of claim 17, wherein the application member comprises a long-latency phosphor.
